(19) United States
(12) Patent Application Publication
(10) Pub. No.: US 2025/0017499 A1
(43) Pub. Date: Jan. 16, 2025

(54) OXYGEN SATURATION SENSOR FOR CLAMPING ATTACHMENT TO A BODY PART

(71) Applicant: Drägerwerk AG & Co. KGaA, Lübeck (DE)

(72) Inventors: Thomas GRAßL, Lübeck (DE); Martha HOOT, Lübeck (DE)

(21) Appl. No.: 18/768,165

(22) Filed: Jul. 10, 2024

(30) Foreign Application Priority Data
Jul. 12, 2023 (DE) 10 2023 118 383.4

(51) Int. Cl. A61B 5/1455; A61B 5/00

(52) U.S. Cl. CPC A61B 5/14552; A61B 5/6838

(57) ABSTRACT

A device for clamping attachment to a body part is provided. The device includes a sensor element, a first clamping element and a second clamping element. The device is adapted to receive the body part between the first clamping element and the second clamping element. The first clamping element includes a first deformable side wall and a second deformable side wall, which form a profile of the first clamping element along a longitudinal axis of the first clamping element. The first clamping element further includes a number of first connecting elements extending between and connecting the first side wall and the second side wall, so that the first clamping element is deformable such that the first clamping element bends towards the body part when applied to the body part.

### CROSS REFERENCE TO RELATED APPLICATIONS

This application claims the benefit of priority under 35 U.S.C. § 119 of German Application 10 2023 118 383.4, filed Jul. 12, 2023, the entire contents of which are incorporated herein by reference.

### TECHNICAL FIELD

The present invention relates to an oxygen saturation sensor for clamping attachment to a body part.

### BACKGROUND

Oxygen saturation sensors for clamping attachment to a body part are known. Oxygen saturation sensors are used to determine the saturation of the blood with oxygen (oxygen saturation). A special design of an oxygen saturation sensor is a pulse oximetric oxygen saturation sensor.

An oxygen saturation sensor is used, for example, in intensive care or in anesthesia medicine, where it is common for a measurement that can be carried out using the oxygen saturation sensor to be performed continuously over a longer period of time.

An oxygen saturation sensor has a light source, for example a light-emitting diode, and a radiation detector, for example a photodiode. To determine the oxygen saturation, a measurement and evaluation of the absorption properties or transmission properties of the body part, for example a user's finger, in relation to the light emitted by the light source.

Depending on the desired application, the oxygen saturation sensor can be reusable or designed for single use.

An oxygen saturation sensor is known from DE 3 703 458 A1. This has a carrier body made of silicone, rubber or polyurethane which can be deformed by material expansion and which can be attached to a finger, arm or leg of a patient and adheres there by clamping forces. The sensor known from DE 3 703 458 A1 is difficult to clean and disinfect due to the complex design of the carrier body.

An oxygen saturation sensor known from DE 69 117 861 T2 has a reusable sensor part which comprises a photodiode and which can be positioned on a patient's finger. The sensor further comprises a disposable flexible element made of non-woven fabric which can be fixed to the finger by an adhesive layer and includes a photoemitter.

### SUMMARY

It is an object of the invention to provide an improved oxygen saturation sensor for clamping attachment to a body part.

These and other problems are solved by the objects of the oxygen saturation sensor according to the invention and by the process for clamping attachment of an oxygen saturation sensor to a body part according to the invention.

This disclosure including the description, claims and drawings present advantageous embodiments of the invention.

According to the invention, an oxygen saturation sensor is provided. The oxygen saturation sensor comprises a first clamping element for receiving a light source or comprising the light source and a second clamping element for receiving the radiation detector or comprising the radiation detector. The oxygen saturation sensor is configured to receive the body part between the first clamping element and the second clamping element. The first clamping element comprises a first, in particular elastically, preferably flexurally, elastic, deformable side wall and a second, preferably elastically deformable, further preferably flexurally elastic, deformable, side wall. The first clamping element further comprises a number of first connecting elements, which extend between the first side wall and the second side wall and preferably connect them, so that the first clamping element is deformable in such a way that the first clamping element bends (curves) in the opposite direction when it is applied to the body part—the first clamping element bends towards the body part as the first clamping element is applied to the body part.

In other words, it is proposed according to the invention to provide a radiation detector for clamping to a body part, which utilizes the fin ray effect, also known as the fin beam effect. The fin ray effect is described in more detail in EP 1 203 640 A2 (EP 1 203 640 A2 is incorporated herein by reference), for example. The fin ray effect has the effect that a fish fin does not bend away in the direction of the force when a lateral force is applied, but rather that the fish fin bends against the direction of the force effect. In the context of the invention, it was recognized that the fin ray effect can be exploited particularly advantageously for an oxygen saturation sensor, since the first clamping element deformed by receiving a body part bends in the direction of the body part and thus rests against the body part and possible pressure points are avoided during prolonged use. This makes it easier to conform an oxygen saturation sensor to the body part or improve the clamping effect on the body part. By placing the first clamping element on the body part, a gap between the body part and the sensor element is also reduced, making it easier to reduce potentially detrimental environmental influences, such as light incidence.

Consequently, counter-bending (counter-curving) is understood to mean that the deformable clamping element deforms concavely when viewed from the direction of an interior of the oxygen saturation sensor, i.e. that at least one end of the first clamping element deforms towards the body location.

Preferably, the oxygen saturation sensor can be locked (latched) in place.

In the context of the invention, an oxygen saturation sensor is understood to mean a device for determining the saturation of the blood with oxygen (oxygen saturation). Preferably, the oxygen saturation sensor is configured as a pulse oximetric oxygen saturation sensor.

According to the invention, the oxygen saturation sensor is configured to be clamped to the body part. The oxygen saturation sensor can be suitably dimensioned for attachment to various body parts suitable for body parts, for example on a finger, an earlobe, an arm and/or a leg, especially of neonates and/or children.

According to the invention, a clamping element refers to an element of the oxygen saturation sensor which is suitable for being brought into clamping contact with the body part of a patient, i.e. an element of the oxygen saturation sensor which provides a contact area for the body part. The first clamping element and the second clamping element thus provide two, in particular opposing, contact areas for the body part.

The clamping elements can be integral with each other or separate from each other and (directly or indirectly) connected to each other.

According to the invention, a side wall refers to a lateral boundary surface of a clamping element, which is preferably a full-surface or alternatively interrupted. Each side wall can have an essentially arbitrary surface shape and can, for example, be flat. A side wall can, for example, be configured to be deformable by selecting a suitable material (in particular elastic or flexurally elastic), for example by one, several or all side walls comprising at least one material selected from the group: thermoplastic elastomer, thermoplastic polyurethane and silicone.

According to the invention, a connecting element refers to an element that extends between two side walls and connects them. A connecting element can, for example, be configured as a bar-shaped (beam-shaped) strut or as an essentially planar (flat or curved) rib. Combinations of these embodiments are also possible. According to the invention, a number of first connecting elements are provided, i.e. only one first connecting element or a plurality of first connecting elements. Each connecting element can be rigid or be configured to be elastically deformable. The connecting element or each connecting element can also be connected to the respective side wall in a flexurally rigid or articulated manner. All that is required is that the clamping element formed by the side walls and the number (one or more) of connecting elements is deformable in such a way that the first clamping element bends (curves) in the opposite direction when it is placed against the body part of these side walls and preferably lies flat against it. It is possible that a strength or a thickness of a connecting element is not constant along at least one direction of extension.

Preferably, the first clamping element and/or the second clamping element is formed through a multi-component injection molding process.

Each clamping element can have additional walls and/or surfaces provided with a material.

The first deformable side wall and the second deformable side wall can form a profile of the first clamping element along a longitudinal axis of the first clamping element. The profile can be essentially triangular, trapezoidal, semicircular, sickle-shaped or polygonal.

The light source can, for example, be a light-emitting diode (hereinafter also referred to as LED or, in the plural, LEDs). Preferably, the oxygen saturation sensor has several light sources, for example a red LED and/or an infrared LED. A red LED is understood to be an LED that is configured to emit light with a wavelength in the range from 610 nm to 760 nm, preferably with a wavelength of 660 nm. An infrared LED is understood to be an LED which is configured to emit light with a wavelength of more than 760 nm, preferably in a range from 800 nm to 1000 nm, particularly preferably with a wavelength of 950 nm.

If the oxygen saturation sensor has several light sources, these can be configured as part of a common component or as structurally separate components.

A radiation detector is a component that is configured to measure electromagnetic radiation, namely light. Such a radiation detector is also referred to as a photodetector. The radiation detector can be, for example, a photo resistor, a photodiode, phototransistor, CCD sensor and/or CMOS sensor. The radiation detector is preferably configured as a photodiode. The oxygen saturation sensor can preferably have several radiation detectors.

Preferably, the light source and the radiation detector are configured as elements on a flexible printed circuit board, which can be formed integrally with the oxygen saturation sensor or which can be removable, i.e. reversibly mounted, in or on the oxygen saturation sensor.

Preferably, the oxygen saturation sensor according to the invention is configured as a disposable oxygen saturation sensor. Alternatively, it is preferred that the oxygen saturation sensor is configured as a reusable oxygen saturation sensor.

Preferably, the first clamping element and the second clamping element are pivotably connected to each other or rigidly connected to each other.

In this way, the design freedom of the oxygen saturation sensor can be increased in terms of the reception of the body part. Rigid connection simplifies the mechanical design and reduces manufacturing costs. A pivotable connection allows the oxygen saturation sensor to be opened for particularly gentle reception of the body part and thus improved comfort.

A pivotable connection can be provided, for example, by an articulated connection of the first clamping element and the second clamping element. For this purpose, the oxygen saturation sensor for example, may have a joint that connects the first clamping element and the second clamping element. Such a joint can, for example, be configured as a swivel joint or a spring joint. In another variant, the first clamping element and the second clamping element can be articulated by means of a material-elastic connection (i.e. a solid-state joint).

The oxygen saturation sensor can also have a spring element that exerts a restoring force on the first clamping element when the relative position of the first clamping element and the second clamping element changes, in order to move the clamping elements back towards their initial (starting) position. This can increase the clamping forces exerted on the body part by the oxygen saturation sensor.

The second clamping element can be of the same or different configuration as that of the first clamping element.

Preferably, the second clamping element is configured as an essentially rigid clamping element, which simplifies the configuration of the oxygen saturation sensor and thus reduces manufacturing costs.

However, it is particularly preferred that the second clamping element has a third, in particular elastically deformable, side wall and a fourth, in particular elastically deformable, side wall. It is preferred that the third side wall and the fourth side wall form a profile of the second clamping element along a longitudinal axis of the second clamping element. It is preferred that the second clamping element has a number (one or more) of second connecting elements which extend between the third side wall and the fourth side wall and preferably connect them, so that the second clamping element is connected to the third side wall that the second clamping element is deformable in such a way that the second clamping element bends in the opposite direction when it is applied to the body part.

In other words, it is particularly preferred that the second clamping element has a fin ray effect as described above. The second clamping element can be configured essentially identically to the first clamping element, which advantageously reduces the variety of parts to be manufactured, or not identically to the first clamping element, whereby a particularly high adaptability of the deformation properties of the oxygen saturation sensor can be achieved.

The profile of the second clamping element can be essentially triangular, trapezoidal, semicircular, sickle-shaped or polygonal.

Preferably, the oxygen saturation sensor is configured such that a difference between an orientation of the light source and radiation detector relative to one another in a deformed state of the respective clamping element and an orientation of the light source and radiation detector relative to one another in a non-deformed state of the respective clamping element is an angular difference of 10°, preferably not exceeding 5°.

Since the light source and radiation detector generally have an angle-dependent radiation or reception characteristic, the signal quality that can be provided by the oxygen saturation sensor can be improved in this way. In other words, in this way, in any deformed state, i.e. irrespective of the size of the body part recorded, it must be ensured that an alignment or orientation of the light source and radiation detector is as parallel as possible to each other (i.e. an amount of an angle (predetermined angle difference) between a central axis of a light-emitting area and a center axis of a light-receiving area is as close as possible to 180° to each other and remains the same in every opening position of the sensor, i.e. for every finger thickness).

A limit to the angular difference required to achieve the best possible signal quality depends primarily on the respective characteristics of the light source and radiation detector. In general, however, it has been recognized that sufficient signal quality can be achieved if the angle difference does not exceed 10°, preferably 5°.

Preferably, the light source is arranged at a distal end of the first clamping element, with the radiation detector preferably being arranged at a distal end of the second clamping element.

A distal end of a clamping element refers to an area of the clamping element that is closer to one end of the clamping element than to a center of the clamping element.

It has been recognized in the context of the invention that a radius of curvature of the clamping element is smaller in a central region than in a distal region of the clamping element, so that in this preferred embodiment a difference between an orientation of the light source and the radiation detector with respect to each other in a deformed state of the respective clamping element and a deformed state of the radiation detector is smaller than in a distal region of the clamping element an orientation of the light source and radiation detector in relation to one another in a non-deformed state of the respective clamping element can be reduced in a particularly simple manner.

Preferably, the first side wall and the second side wall enclose a first acute angle in an undeformed state of the first clamping element. Additionally or alternatively, it is preferred that the third side wall and the fourth side wall enclose a second acute angle in an undeformed state of the second clamping element.

This improves the deformability of the first clamping element and/or the second clamping element and thus the attachment to the body part.

In the event that the side walls are not flat, the acute angle is enclosed by respective tangents to the respective side walls.

Preferably, a portion of the number of first connecting elements and/or a portion of the number of second connecting elements is of a planar configuration, with respective surface normals of the connecting elements being arranged perpendicular to the respective longitudinal axis of the respective clamping element.

In this way, an advantageous symmetrical deformation of the first clamping element and/or the second clamping element can be achieved. Furthermore, the deformability of the respective clamping element in the direction of the body part is improved in this way and the respective clamping element is stiffened against deformation in the opposite direction.

Particularly preferably, the total number of first connecting elements and/or the total number of second connecting elements is configured in the form of a surface, with respective surface normals of the connecting elements being arranged perpendicular to the respective longitudinal axis of the respective clamping element.

Preferably, the first side wall and/or the third side wall is provided by side wall segments spaced apart from one another and/or by overlapping side wall segments.

In this way, cavities formed between the respective connecting elements and side wall segments can be made accessible for cleaning, which improves the cleanability of the oxygen saturation sensor.

Preferably, the first side wall and/or the second side wall and/or the third side wall and/or the fourth side wall are configured to be interrupted transversely to the direction of the respective longitudinal axis of the respective clamping element.

In this way, the contact of the oxygen saturation sensor with the body part can be improved and, in particular, the incidence of unwanted light from the environment in the direction of the radiation detector can be reduced.

The oxygen saturation sensor can also have further elements, for example for data processing and communication. For example, the oxygen saturation sensor can have a control unit such as a circuit or a microprocessor for controlling the light source and/or the oxygen saturation sensor radiation detector. The control unit can, for example, be configured to receive and process measurement signals from the radiation detector and/or store them in a memory unit. Furthermore, for example, the oxygen saturation sensor can have circuits for controlling and/or reading out the light source and/or the radiation detector. The circuits can, for example, have one or more amplifiers, resistors, capacitors, filters, A/D converters and the like. The circuits can be configured as part of the control unit or as separate components. Furthermore, for example, the oxygen saturation sensor can have a data interface, for example have a wired or wireless interface for sending and/or receiving data. For example, the interface can be used to transmit the measurement signals of the radiation detector and/or measurement data stored in a memory unit to an external receiver. Furthermore, for example, the oxygen saturation sensor may have an energy source such as an accumulator or a battery to provide electrical energy for the operation of the oxygen saturation sensor. In addition or alternatively, the oxygen saturation sensor can have a power interface (wired or wireless) for receiving electrical energy for operating the oxygen saturation sensor. oxygen saturation sensor. All of the aforementioned elements can be configured as part of a common printed circuit board, for example as part of the flexible printed circuit board or printed circuit boards described above.

Furthermore, the above-mentioned task is solved by a process using a fin ray structure in an oxygen saturation sensor for clamping attachment to a body part.

These and other features and advantages can also be seen from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a perspective view showing an embodiment of an oxygen saturation sensor according to the invention;

FIG. 1b is a perspective view showing an embodiment of the oxygen saturation sensor according to the invention as shown in FIG. 1a in a state attached to a body part;

FIG. 2a is a perspective view showing an embodiment of a first clamping element according to the invention;

FIG. 2b is a front side view showing the first clamping element according to the invention as shown in FIG. 2a;

FIG. 2c is a front side view showing an embodiment of a first clamping element according to the invention;

FIG. 2d is a front side view showing an embodiment of a first clamping element;

FIG. 2e is a front side view showing an embodiment of a first clamping element according to the invention;

FIG. 2f is a front side view showing an embodiment of a first clamping element according to the invention;

FIG. 3a is a front side partially schematic view showing an embodiment of an oxygen saturation sensor;

FIG. 3b is a front side partially schematic view showing an embodiment of the oxygen saturation sensor according to the invention, as shown in FIG. 3a, in a state attached to a body part;

FIG. 3c is a front side partially schematic view showing an embodiment of an oxygen saturation sensor according to the invention;

FIG. 4 is a front side view showing an embodiment of an oxygen saturation sensor in a deformed state;

FIG. 5a is a perspective view showing an embodiment of an oxygen saturation sensor according to the invention;

FIG. 5b is a perspective view showing an embodiment of the oxygen saturation sensor according to the invention, as shown in FIG. 5a, in an open state;

FIG. 6a is a perspective view showing an embodiment of an oxygen saturation sensor;

FIG. 6b is a perspective view showing an embodiment of the oxygen saturation sensor according to the invention, as shown in FIG. 6a, in an open state;

FIG. 6c is a perspective view showing an embodiment of the oxygen saturation sensor according to the invention, as shown in FIG. 6a and FIG. 6b, in a state attached to a body part;

FIG. 7 is a perspective view showing an embodiment of an oxygen saturation sensor according to the invention;

FIG. 8 is a perspective view showing an embodiment of an oxygen saturation sensor according to the invention; and

FIG. 9 is a perspective view showing an embodiment of an oxygen saturation sensor according to the invention.

### DESCRIPTION OF PREFERRED EMBODIMENTS

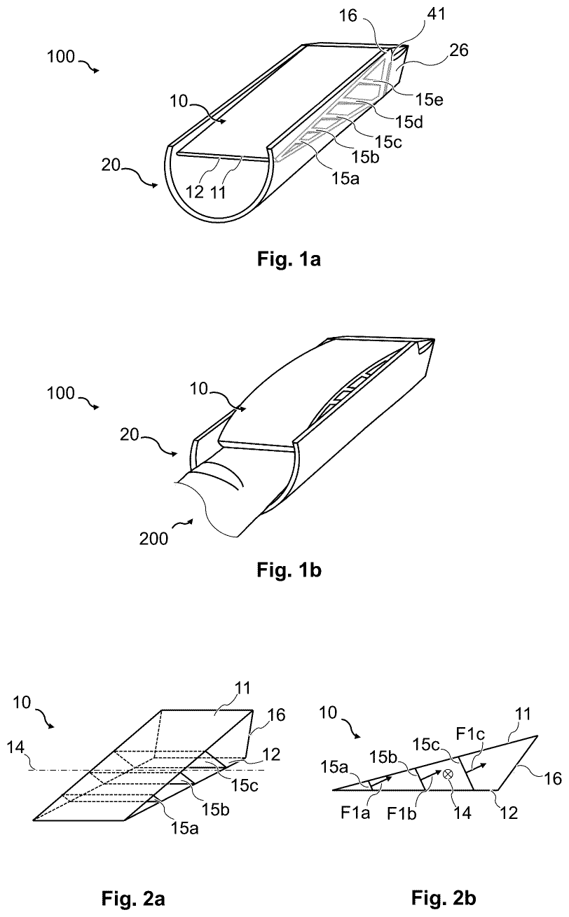

Referring to the drawings, the present invention relates to an oxygen saturation sensor 100 for attachment to a body part 200, for example to a finger 200. Embodiments of oxygen saturation sensors 100 according to the invention or components thereof are shown in FIGS. 1-9.

All embodiments of oxygen saturation sensors 100 according to the invention comprise a first clamping element 10 and a second clamping element 20, wherein the oxygen saturation sensors 100 are arranged to receive the body part 200 between the first clamping element 10 and the second clamping element 20.

All embodiments of the first clamping element 10 comprise a first deformable side wall 11 and a second deformable side wall 12, wherein these can form a profile of the first clamping element 10 along a longitudinal axis 14 of the first clamping element 10.

All embodiments of the first clamping element 10 further comprise a number (one or more) of first connecting elements 15a, 15b, 15c, . . . , which extend between the first side wall 11 and the second side wall 12 and preferably connect them.

In all illustrated embodiments of the first clamping element 10, the number of connecting elements 15a, 15b, 15c, . . . is formed as a plurality. In embodiment examples not shown, the number of connecting elements 15 can be configured as a single number, i.e. the first clamping element 10 can also have only one connecting element 15, although this is less preferred.

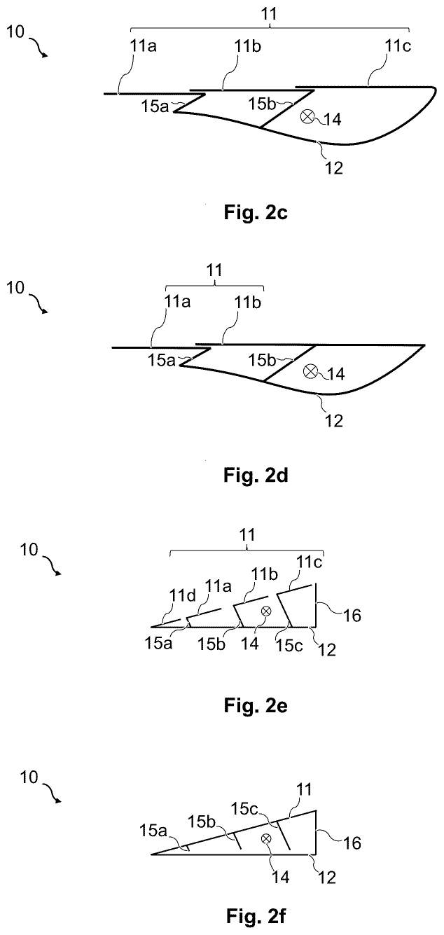

In some embodiments, the connecting elements 15a, 15b, 15c, . . . is formed as a surface, whereby the respective surface normals F1 of the connecting elements 15a, 15b, 15c, . . . are arranged perpendicular to the longitudinal axis 14 of the respective clamping element 10. For the sake of clarity, the surface normals F1 are only shown in FIG. 2b and are omitted in the other figures. However, in embodiments not shown, only a part of the number (one or more) of connecting elements 15a, 15b, 15c, . . . can also be configured in the form of a surface. In further embodiments not shown, the number of connecting elements 15a, 15b, 15c, . . . may also not be in the form of a surface but, for example, in the form of a bar (beam). Although the illustrated connecting elements 15a, 15b, 15c, . . . have a flat surface shape, non-flat connecting elements 15a, 15b, 15c, . . . are also possible.

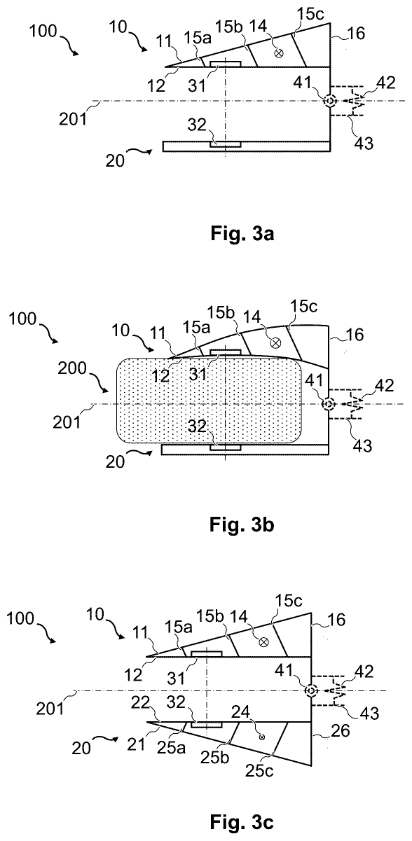

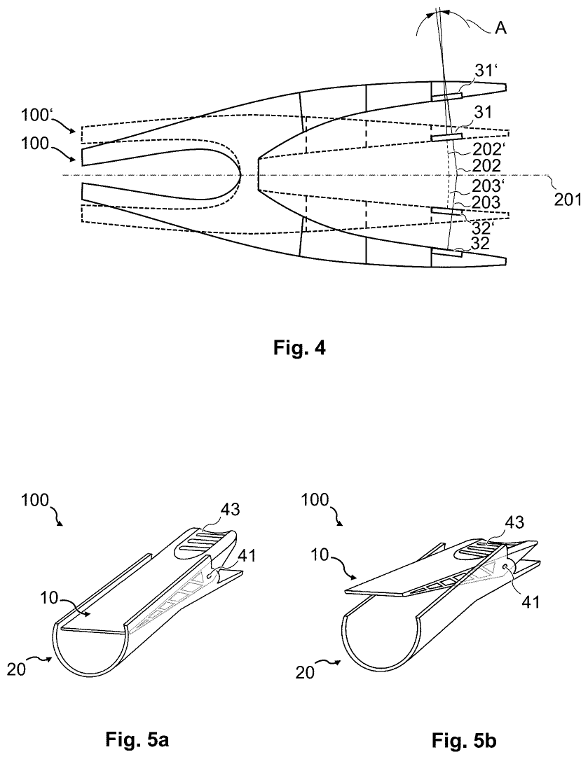

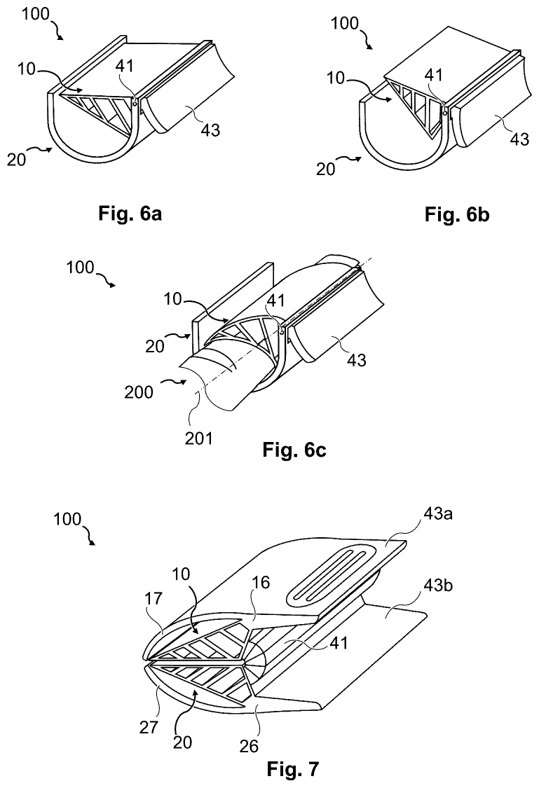

In all embodiments of the oxygen saturation sensor 100 according to the invention, the first clamping element 10 is deformable in such a way that the first clamping element 10 bends towards the body 200 when the first clamping element 10 is applied to the body 200. This feature is illustrated in the comparison between the respective undeformed state of the first clamping element 10 illustrated in FIG. 1a, FIG. 3a, FIG. 4 and FIG. 6a and FIG. 6b in comparison with the respective deformed state of the first clamping element 10 illustrated in FIG. 1b, FIG. 3b, FIG. 4 and FIG. 6c.

The oxygen saturation sensor 100 according to the invention thus has a first clamping element 10, which has a fin ray effect.

In the following, the oxygen saturation sensors 100 are described in more detail according to the individual embodiment examples.

The oxygen saturation sensor 100 according to FIGS. 1a, 1b has, in addition to the first clamping element 10, a second clamping element 20, which is concave to accommodate the body part 200. Furthermore, the oxygen saturation sensor 100 has a light source 31 and a radiation detector 32, which are not shown for the sake of clarity.

The first clamping element 10 and the second clamping element 20 are pivotably connected to one another by a joint 41, in this embodiment example by a solid joint 41 (a joint formed of the material connection between the first clamping element 10 and the second clamping element 20). Alternatively, the joint 41 can be configured as a separate (joint) component. The first clamping element 10 has a first side wall 11 and a second side wall 12 as well as five connecting elements 15a-15e, which extend between the first side wall 11 and the second side wall 12 and preferably connect the first side wall 11 and the second side wall 12. However, the number of connecting elements 15a, 15b, . . . can be configured differently from this. The first clamping element 10 also has a first base wall 16, which is connected to a second base wall 26 of the second clamping element 20 by the joint 41. However, a base wall 16 is not required. The first clamping element 10 and the second clamping element 20 can be integrally formed with one another, whereby the first clamping element 10 bends towards the body 200 when the first clamping element 10 is placed against the body 200 in order to accommodate the body 200, which is shown as a finger 200 in FIG. 1b. In the illustrated embodiment example, the first side wall 11 and the second side wall 12 form a profile of the first clamping element 10 along a longitudinal axis 14 of the oxygen saturation sensor 100 first clamping element 10.

The first clamping element 10 according to this embodiment can in principle be combined with any other embodiment of an oxygen saturation sensor 100. Likewise, each second clamping element 20 may have a configuration that is the same or similar to the first clamping element 10 as just described.

The oxygen saturation sensor 100 according to FIGS. 5a, 5b corresponds to the oxygen saturation sensor 100 according to FIGS. 1a, 1b except for the differences described below, so that the similarities are not repeated. In contrast to the oxygen saturation sensor 100 according to FIGS. 1a, 1b, the oxygen saturation sensor 100 according to FIGS. 5a, 5b has a separate joint 41, which pivotably connects the first clamping element 10 and the second clamping element 20. The oxygen saturation sensor 100 according to FIGS. 5a, 5b also has an actuation mechanism 43 by means of which the first clamping element 10 and the second clamping element 20 can be moved relative to one another to receive the body part 200. Such an actuation mechanism 43 can be used in any embodiment of an oxygen saturation sensor 100.

In FIGS. 2a, 2b, 2c, 2d, 2e, 2f, five embodiments of a first clamping element 10 are shown, which can be combined with any embodiment of an oxygen saturation sensor 100. Likewise, each second clamping element 20 may have a configuration that is the same or similar to the first clamping element 10 as just described.

The first clamping element 10 according to FIGS. 2a, 2b essentially corresponds to the first clamping element 10 according to FIGS. 1a, 1b, although in contrast it only has three connecting elements 15a, 15b, 15c. It is possible in every exemplary embodiment, but only shown explicitly in FIGS. 2a, 2b, that part or all of the number of first connecting elements 15a, 15b, . . . may be configured to be planar, with respective surface normals F1 of the connecting elements 15a, 15b, . . . , arranged perpendicular to the respective longitudinal axis 14 of the respective clamping element 10, 20. An alternative to the planar design of part or all of the number of first connecting elements 15a, 15b, . . . is to configure part or all of the number of first connecting elements 15a, 15b in a bar shape.

The second clamping element 20 may have a configuration that is the same or similar to the first clamping element 10 as just described.

The first clamping element 10 according to FIG. 2c and the first clamping element 10 according to FIG. 2d differ from the first clamping elements 10 described above in that their first side wall 11 is provided by overlapping side wall segments 11a, 11b, 11c. As shown in FIG. 2d, it is possible, for example, that the number of connecting elements 15a, 15b corresponds to the number of side wall segments 11a, 11b. However, as shown in FIG. 2c, it is also possible for the number of connecting elements 15a, 15b to be different from the number of side wall segments 11a, 11b, 11c.

The second clamping element 20 may have a configuration that is the same or similar to the first clamping element 10 as just described.

The first clamping element 10 according to FIG. 2e differs from the pre-described first clamping elements 10 in that their first side wall 11 is provided by side wall segments 11a, 11b, 11c, 11d spaced apart from one another. It is possible that the number of connecting elements 15a, 15b corresponds to the number of side wall segments 11a, 11b. However, as shown in FIG. 2e, it is also possible that the number of connecting elements 15a, 15b is different from the number of side wall segments 11a, 11b, 11c.

The second clamping element 20 may have a configuration that is the same or similar to the first clamping element 10 as just described.

The first clamping element 10 according to FIG. 2f differs from the first clamping elements 10 described above in that the number of connecting elements 15a, 15b, 15c does not directly connect the first side wall 11 and the second side wall 12.

The second clamping element 20 may have a configuration that is the same or similar to the first clamping element 10 as just described.

In addition to a first clamping element 10, an oxygen saturation sensor 100 shown schematically in FIGS. 3a, 3b has a second clamping element 20, which can be configured to accommodate the body part 200. Such a configuration of the second clamping element 20 is possible in all embodiments of an oxygen saturation sensor 100. The first clamping element 10 and the second clamping element 20 of this embodiment are pivotably connected to each other by a joint 41. The joint 41 can be configured as a solid hinge (solid joint) or as a joint component. The first clamping element 10 can be a previously described first clamping element 10 or may be another first clamping element 10. The oxygen saturation sensor 100 also optionally has a spring 42 as a spring element, which exerts a restoring force on the first clamping element 10 and the second clamping element 20 when the relative position of the first clamping element 10 and the second clamping element 20 to each other changes, in order to move them back in the direction of their initial position. In each embodiment example, it is preferred that the light source 31 and the sensor element 32 lie with their respective center axis in a common plane, as indicated by the dash-dot line in FIGS. 3a, 3b.

The oxygen saturation sensor 100 according to FIGS. 3a, 3b is also suitable for clamping attachment to a body part 200, which is shown schematically in FIG. 3b. For example, the longitudinal axis 14 of the first clamping element 10 can be aligned transversely to a longitudinal direction 201 of the body site in a state attached to the body site 200, as indicated in FIG. 3b. In other embodiments, as shown for example in FIGS. 6a, 6b, 6c, the longitudinal axis 14 of the first clamping element 10 can be aligned parallel or longitudinally to a longitudinal direction 201 of the body site in a state attached to the body site 200.

The oxygen saturation sensor 100 according to FIG. 3c corresponds to the oxygen saturation sensor 100 according to FIGS. 3a, 3b except for the differences described below, so that the similarities are not repeated. In contrast to the oxygen saturation sensor 100 according to FIGS. 3a, 3b, the oxygen saturation sensor 100 according to FIG. 3c also has a second clamping element 20, which is formed in the manner of a fin ray structure, instead of a flat second clamping element 20. In other words, the second clamping element 20 according to FIG. 3c comprises a third deformable side wall 21 and a fourth deformable side wall 22, which preferably form a profile of the second clamping element 20 along a longitudinal axis 24 of the second clamping element 20, and further comprises a number (one or more) of second connecting elements 25, which extend between the third side wall 21 and the fourth side wall 22 and preferably connect them, so that the second clamping element 20 is deformable in such a way that the second clamping element 20 bends towards the body part 200 when the second clamping element 20 is applied to the body part 200. Such a second clamping element 20 can be provided in any embodiment of an oxygen saturation sensor 100.

FIG. 4 shows a modification of the oxygen saturation sensor 100 shown in FIG. 3c. The oxygen saturation sensor 100 with solid lines represents a state deformed by receiving a body part 200 (not shown). The oxygen saturation sensor 100′ with broken line represents a non-deformed state. The respective orientation of light source 31′ in the deformed state and of light source 31 in the non-deformed state is characterized by a normal 202′ or 202, respectively. The respective orientation of radiation detector 32′ in the deformed state and radiation detector 32 in the non-deformed state is characterized by a normal 203′ or 203. It is desirable that a difference between the orientation (normal 202) of light source 31 and radiation detector 32 (normal 203) relative to each other in a deformed state and an orientation (normal 202′) of light source 31′ and radiation detector 32′ (normal 203′) relative to each other in a non-deformed state does not exceed an angular difference A of 10°.

The oxygen saturation sensor 100 according to FIGS. 6a, 6b and 6c has, in addition to the first clamping element 10, a second clamping element 20 which is concave in shape to accommodate the body part 200. Such a second clamping element 20 can be provided in any embodiment of an oxygen saturation sensor 100. The first clamping element 10 and the second clamping element 20 are pivotably connected to each other by a joint 41. In this embodiment example, the longitudinal axis of the first clamping element 10 and a longitudinal axis of the second clamping element 20 are aligned, which can run along the profile of the second clamping element 20, are aligned parallel to one another. The oxygen saturation sensor 100 also has an actuation mechanism 43 and preferably also a spring element (not shown).

The oxygen saturation sensor 100 according to FIG. 7 corresponds to the oxygen saturation sensor 100 according to FIG. 3c except for the differences described below, so that the similarities will not be repeated. In contrast to the oxygen saturation sensor 100 shown in FIG. 3c, the oxygen saturation sensor 100 according to FIG. 7 also has a second clamping element 20, which has a fin ray effect, instead of a flat second clamping element 20. The first clamping element 10 and the second clamping element 20 of this embodiment are connected by a solid joint 41, which also acts as a spring element and provides the aforementioned restoring force. Likewise, the oxygen saturation sensor 100 according to this embodiment has a two-part actuation mechanism 43a, 43b. Finally, the oxygen saturation sensor 100 according to this embodiment has a first cover 17 and a second cover 27. Such an embodiment of the solid joint 41 and/or a two-part actuation mechanism 43a, 43b can be provided in any embodiment of an oxygen saturation sensor 100.

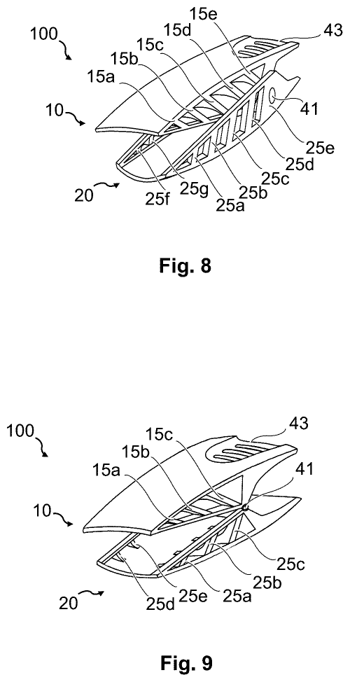

In variants of the invention, the first side wall 11 and/or the second side wall 12 and/or the third side wall 21 and/or the fourth side wall 22 can be configured to be interrupted, in particular in order to accommodate the body part 200. This is shown in the embodiments according to FIG. 8 and FIG. 9, in which the side wall lying on an inner side of the oxygen saturation sensor 100 in each case is configured to be interrupted.

The oxygen saturation sensor 100 according to FIG. 8 has the first clamping element 10 with a plurality of connecting elements 15a-15j, which are located in the first clamping element 10 essentially in the form of a bar or can extend in the form of an interrupted surface in the direction of the longitudinal axis 14 of the first clamping element 10. This provides an interruption between the two base sides of the first clamping element 10, in which the body part 200 can be accommodated. The second clamping element 20 is configured in the same way as the first clamping element 10, and thus also has a plurality of connecting elements 25a-25j, which are essentially bar-shaped or can extend in the form of an interrupted surface in the direction of the longitudinal axis 24 of the second clamping element 20. The first clamping element 10 and the second clamping element 20 can optionally be pivotably connected to one another by a joint 41. The oxygen saturation sensor 100 can also optionally have an actuation mechanism 43. It is preferred and shown in FIG. 8 that the first clamping element 10 and the second clamping element 20 can be configured to engage (interlock) with one another, so that the first clamping element 10 and the second clamping element 20 overlap when viewed in the longitudinal direction 14 of the first clamping element 10. Such a configuration of the first clamping element 10 and/or the second clamping element 20 can be provided in any embodiment of an oxygen saturation sensor 100.

The oxygen saturation sensor 100 according to FIG. 9 has the first clamping element 10 with a plurality of connecting elements 15a-15f, which are essentially bar-shaped or extend interrupted in the direction of the first clamping element of the longitudinal axis 14 of the first clamping element 10. This provides an interruption between the two base sides of the first clamping element 10, in which the body part 200 can be accommodated. The second clamping element 20 is configured in the same way as the first clamping element 10, i.e. the second clamping element 20 also has a plurality of connecting elements 25a-25f, which are essentially bar-shaped or can extend in the form of an interrupted surface in the direction of the longitudinal axis 24 of the second clamping element 20. The first clamping element 10 and the second clamping element 20 can optionally be pivotably connected to each other by a joint 41. The oxygen saturation sensor 100 can furthermore optionally have an actuation mechanism 43. Such a configuration of the first clamping element 10 and/or the second clamping element 20 may be provided in any embodiment of an oxygen saturation sensor 100.

All of the options described herein for providing the oxygen saturation sensor can be combined with each other as desired, provided that this is not contradictory or does not involve alternatives.

Instead of an oxygen saturation sensor as described above, all features of the invention can also be provided by a device for clamping attachment to a body part for measuring medically relevant data. An example of such a device is a pre-described oxygen saturation sensor.

While specific embodiments of the invention have been shown and described in detail to illustrate the application of the principles of the invention, it will be understood that the invention may be embodied otherwise without departing from such principles.

### LIST OF REFERENCE CHARACTERS

10 First clamping element
11 First side wall
12 Second side wall
14 Longitudinal axis of the first clamping element
15, 15a, 15b, . . . . First connecting element(s)
16 First base wall
17 First cover
20 Second clamping element
21 Third side wall (panel)
22 Fourth side wall (panel)
24 Longitudinal axis of the second clamping element
25, 25a, 25b, . . . . Second connecting element(s)
26 Second base wall
27 Second cover
31, 31′ Light source
32, 32′ Radiation detector
41 Joint
42 Spring
43, 43a, 43b Actuation mechanism
100 Oxygen saturation sensor
200 Body part, finger
201, 201′ Longitudinal direction of the body part
202, 202′ Center axis/normal of the light source
203, 203′ Center axis/normal of the radiation detector
A, Angle difference
F1, F1a, F1b, . . . . Surface normal of the first connecting element(s)

What is claimed is:

1. An oxygen saturation sensor for clamping attachment to a body part, the oxygen saturation sensor comprising:
a first clamping element configured to receive a light source or comprising the light source; and
a second clamping element configured to receive a radiation detector or comprising the radiation detector,
wherein the oxygen saturation sensor is configured to receive the body part between the first clamping element and the second clamping element, and
wherein the first clamping element comprises:
a first deformable side wall;
a second deformable side wall; and
a number of first connecting elements, which extend between the first side wall and the second side wall,
wherein the first clamping element is configured to be deformable such that the first clamping element bends towards the body part upon the first clamping element being applied to the body part.

2. An oxygen saturation sensor according to claim 1, wherein the first clamping element and the second clamping element are pivotably connected to one another or rigidly connected to one another.

3. An oxygen saturation sensor according to claim 1, wherein the second clamping element comprises:
a third deformable side wall;
a fourth deformable side wall, and
a number of second connecting elements, which are located between the third side wall and the fourth side wall and extend, wherein the second clamping element is configured to be deformable such that the second clamping element bends towards the body part upon the the second clamping element being applied to the body part.

4. An oxygen saturation sensor according to claim 1, wherein the oxygen saturation sensor is configured such that a difference between an orientation of the light source and the radiation detector relative to each other in a deformed state of the first clamping element and an orientation of light source and radiation detector in relative to each other in a non-deformed state of the first clamping element does not exceed an angular difference of 10°.

5. An oxygen saturation sensor according to claim 3, wherein the oxygen saturation sensor is configured such that a difference between an orientation of the light source and the radiation detector relative to each other in a deformed state of the respective clamping element and an orientation of light source and radiation detector in relative to each other in a non-deformed state of the respective clamping element does not exceed an angular difference of 10°.

6. An oxygen saturation sensor according to claim 4, wherein the light source is arranged at a distal end of the first clamping element, and wherein the radiation detector is arranged at a distal end of the second clamping element.

7. An oxygen saturation sensor according to claim 1, wherein the first side wall and the second side wall enclose an acute angle in an undeformed state of the first clamping element.

8. An oxygen saturation sensor according to claim 3,
wherein the first side wall and the second side wall enclose an acute angle in an undeformed state of the first clamping element, and/or
wherein the third side wall and the fourth side wall enclose an acute angle in an undeformed state of the second clamping element.

9. An oxygen saturation sensor according to claim 1,
wherein at least one of the first connecting elements is planar, and
wherein a surface normal of the at least one of the first connecting elements is arranged perpendicular to a longitudinal axis of the first clamping element.

10. An oxygen saturation sensor according to claim 3,
wherein at least one of the first connecting elements is planar and/or at least one of the second connecting elements is planar, and
wherein a surface normal of the at least one of the first connecting elements is arranged perpendicular to a longitudinal axis of the first clamping element and/or a surface normal of the at least one of the second connecting elements is arranged perpendicular to a longitudinal axis of the second clamping element.

11. An oxygen saturation sensor according to claim 1, wherein the first side wall is provided by side wall segments spaced apart from each other and/or by overlapping side wall segments.

12. An oxygen saturation sensor according to claim 3, wherein the first side wall and/or the third side wall is provided by side wall segments spaced apart from each other and/or by overlapping side wall segments.

13. An oxygen saturation sensor according to claim 1, wherein the first side wall and/or the second side wall is interrupted transversely to a direction of a longitudinal axis of the first clamping element.

14. An oxygen saturation sensor according to claim 3, wherein the first side wall and/or the second side wall and/or the third side wall and/or the fourth side wall is interrupted transversely to a direction of a respective longitudinal axis of the respective clamping element.

15. An oxygen saturation sensor according to claim 1 wherein the first clamping element comprises a fin ray structure.

16. A process for clamping attachment of an oxygen saturation sensor to a body part, the process comprising the steps of:
providing the oxygen saturation sensor, wherein the oxygen saturation sensor comprises: a first clamping element configured to receive a light source or comprising the light source; and a second clamping element configured to receive a radiation detector or comprising the radiation detector, wherein the first clamping element comprises: a first deformable side wall; a second deformable side wall; and a first connecting element, which extends between the first side wall and the second side wall;
configuring the oxygen saturation sensor to receive the body part between the first clamping element and the second clamping element; and
configuring the first clamping element to be deformable such that the first clamping element bends towards the body part upon the first clamping element being applied to the body part; and
applying the first clamping element to the body part with the first clamping element bending towards the body part with the first clamping element applied to the body part.

17. A process according to claim 16, wherein the first clamping element comprises a fin ray structure which is used to bend the first clamping element towards the body part with the first clamping element applied to the body part.

18. An oxygen saturation sensor comprising:
a first clamping element comprising: a first deformable side wall; a second deformable side wall; and a connecting element, which extends between the first side wall and the second side wall, wherein the first clamping element is configured to be deformable to bend towards the body part upon the first clamping element being applied to the body part;
a second clamping element, wherein the first clamping element and the second clamping element are together configured to receive the body part between the first clamping element and the second clamping element;
a light source connected to one of the first clamping element and the second clamping element; and
a radiation detector connected to another of the first clamping element and the second clamping element.

19. An oxygen saturation sensor according to claim 18, wherein the second clamping element comprises:
a third deformable side wall;
a fourth deformable side wall, and
a second clamping element connecting elements located between the third side wall and the fourth side wall, wherein the second clamping element is configured to be deformable to bend towards the body part upon the second clamping element being applied to the body part.

20. An oxygen saturation sensor according to claim 18, wherein the oxygen saturation sensor is configured such that a difference between an orientation of the light source and the radiation detector relative to each other in a deformed state of the first clamping element and an orientation of light source and radiation detector relative to each other in a non-deformed state of the first clamping element does not exceed an angular difference of 10°.
